(19) United States
(12) Patent Application Publication
(10) Pub. No.: US 2026/0362964 A1
(43) Pub. Date: Nov. 12, 2026

(54) ORGANIC LIGHT-EMITTING DIODE DEVICE

(71) Applicant: JAPAN SCIENCE AND TECHNOLOGY AGENCY, Saitama (JP)

(72) Inventors: Seiichiro IZAWA, Tokyo (JP); Yutaka MAJIMA, Tokyo (JP); Masahiro MORIMOTO, Toyama (JP); Shigeki NAKA, Toyama (JP)

(73) Assignee: JAPAN SCIENCE AND TECHNOLOGY AGENCY, Saitama (JP)

(21) Appl. No.: 19/702,935

(22) Filed: Jun. 9, 2026

Related U.S. Application Data
(63) Continuation of application No. PCT/JP2025/002089, filed on Jan. 23, 2025.

(30) Foreign Application Priority Data
Jan. 31, 2024 (JP) 2024-012785

(51) Int. Cl. H10K 85/60; C09K 11/06; H10K 50/12; H10K 101/30

(52) U.S. Cl. CPC H10K 85/622; C09K 11/06; H10K 85/624; H10K 85/626; H10K 85/6572; C09K 2211/1007; C09K 2211/1011; H10K 50/12; H10K 2101/30

(57) ABSTRACT

An OLED device 10 including a layered structure sandwiched between a pair of electrodes 3 and 4, wherein the layered structure has an acceptor layer 1 and a donor layer 2 forming an interface with the acceptor layer 1, the acceptor layer 1 contains an acceptor molecule, the donor layer 2 contains a donor molecule that causes triplet-triplet annihilation, and the acceptor molecule and the donor molecule satisfy requirements and the like relating to predetermined energy levels.

### TECHNICAL FIELD

The present invention relates to an OLED device.

### BACKGROUND ART

An organic electroluminescence device (OLED device) is a device that includes one or two or more organic semiconductor layers sandwiched between a pair of electrodes, and emits light by itself when a voltage is applied between the electrodes.

In recent years, research has been actively conducted to increase light-emission efficiency and luminance of OLED devices. For example, the present inventors have proposed an OLED device which has a predetermined configuration and is excellent in light-emission efficiency and luminance (refer to Patent Literature 1).

### CITATION LIST

### Patent Literature

[Patent Literature 1] International Publication No. 2022/211041

### SUMMARY OF INVENTION

### Technical Problem

However, as a result of studies conducted by the present inventors, it has become clear that there is room for improvement in light-emission efficiency and luminance with respect to conventional OLED devices such as those in Patent Literature 1.

An object of the present invention is therefore to provide an OLED device excellent in light-emission efficiency and luminance.

### Solution to Problem

The present inventors have conducted a thorough examination in consideration of the above-described circumstances, and as a result thereof, they found an OLED device having the following configuration.

That is, the OLED device of the present invention is,
an OLED device including a pair of electrodes and a layered structure sandwiched between the electrodes,
wherein the layered structure has an acceptor layer and a donor layer forming an interface with the acceptor layer,
the acceptor layer contains an acceptor molecule,
the donor layer contains a donor molecule that causes triplet-triplet annihilation,
a HOMO level of the acceptor molecule is lower than a HOMO level of the donor molecule, and a LUMO level of the acceptor molecule is lower than a LUMO level of the donor molecule,
an excited triplet level of the donor molecule is smaller than a value obtained by adding 0.1 eV to an energy difference between the HOMO level of the donor molecule and the LUMO level of the acceptor molecule,
an excited triplet level of the acceptor molecule is larger than the energy difference between the HOMO level of the donor molecule and the LUMO level of the acceptor molecule, and
the energy difference between the HOMO level of the donor molecule and the LUMO level of the acceptor molecule is smaller than an energy difference between the HOMO level and the LUMO level of the donor molecule by 0.4 eV or more.

However, a case may be excluded where the acceptor layer is composed of an acceptor molecule represented by the following formula (A), the donor layer is composed of a first layer forming an interface with the acceptor layer and a second layer laminated on the first layer, the first layer is composed of a donor molecule represented by the following formula (B) and a dopant molecule represented by the following formula (C), and the second layer is composed of a donor molecule represented by the following formula (B); a case may be excluded where the acceptor layer is composed of an acceptor molecule represented by the following formula (A), and the donor layer (the donor layer may be a single layer or may be composed of a plurality of layers) is composed of a donor molecule represented by the following formula (B) and a dopant molecule represented by the following formula (C); a case may be excluded where the acceptor layer is composed of an acceptor molecule represented by the following formula (A), and the donor layer (the donor layer may be a single layer or may be composed of a plurality of layers) contains a donor molecule represented by the following formula (B); and a case may be excluded where the acceptor layer is composed of an acceptor molecule represented by the following formula (A).

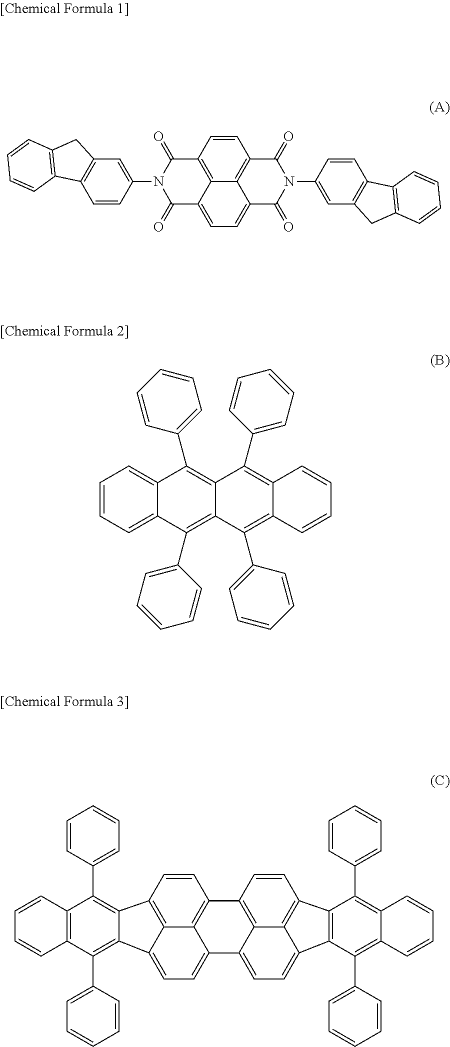

According to such an OLED device, light-emission efficiency and luminance are excellent. The reason for this is not necessarily clear, but consideration made by the present inventors will be described with reference to FIGS. 1, 2, 3A and 3B.

FIG. 1 is a conceptual diagram illustrating a mechanism in which light-emission is caused to occur by the OLED device of the present invention. In FIG. 1, an OLED device 10 has an acceptor layer 1, a donor layer 2 forming an interface with the acceptor layer 1, a first electrode 3 formed on the acceptor layer 1 side, and a second electrode 4 formed on the donor layer 2 side. In the explanation stated here, respectively, the acceptor layer 1 corresponds to an electron transport layer, the donor layer 2 corresponds to a light-emitting layer, the first electrode 3 corresponds to a cathode, and the second electrode 4 corresponds to an anode, as in the case of Examples.

When electrons (−) are injected to the OLED device 10 from the cathode and holes (+) are injected to the OLED device 10 from the anode, an electron (−)/hole (+) pair forms a charge transfer (CT) state at the interface between the acceptor layer 1 and the donor layer 2. A triplet state T_{1D} of the donor molecule is generated in the donor layer 2 by charge recombination of the CT state. A high-energy excited state (S₁) is generated by causing triplet-triplet annihilation (TTA) to occur in the donor layer 2. In a case where no dopant molecule is present in the donor layer 2, light-emission derived from the donor molecule occurs. In a case where a dopant molecule is present in the donor layer 2, energy transfer from the donor molecule to the dopant molecule occurs, whereby light-emission derived from the dopant molecule occurs.

FIG. 2 is a view illustrating energy levels of rubrene, perylene, NDI-HF, and NDI-Cy used in Examples. NDI-HF and NDI-Cy correspond to acceptor molecules, and rubrene and perylene correspond to donor molecules, respectively. Since the HOMO level of the acceptor molecule is lower than the HOMO level of the donor molecule, the LUMO level of the acceptor molecule is lower than the LUMO level of the donor molecule, and the energy difference between the HOMO level of the donor molecule and the LUMO level of the acceptor molecule is smaller than the energy difference between the HOMO level and the LUMO level of the donor molecule by 0.4 eV or more, it is considered that the electron (−)/hole (+) pair injected from the electrodes can form the CT state at the interface between the acceptor layer 1 and the donor layer 2.

FIG. 3A is a schematic diagram illustrating an energy transfer mechanism leading to light-emission by the OLED device of the present invention. The energy difference between the HOMO level of the donor molecule and the LUMO level of the acceptor molecule corresponds to the energy level of the CT₃ state (hereinafter, also simply referred to as “CT”). In FIG. 3A, the excited triplet level TIA of the acceptor molecule of the OLED device of the present invention is larger than CT (for example, in the case of T_{1A′} in the figure), and the excited triplet level T_{1D} of the donor molecule is smaller than the value obtained by adding 0.1 eV to CT.

In contrast, as illustrated in FIG. 3B, in a case where the excited triplet level of the acceptor molecule is smaller than CT (for example, in the case of T_{1A″} in the figure), energy efficiency is inferior compared to that of the OLED device of the present invention. The reason for this is that, as illustrated in FIG. 3B, there exists a route by which energy transition from the CT state to the excited triplet state T_{1D} of the donor molecule and energy transition to the excited triplet state T_{1A″} of the acceptor molecule compete with each other, and the system returns to the ground state S₀ of the acceptor via non-radiative deactivation.

On the other hand, as illustrated in FIG. 3A, in the case of T_{1A′} where the excited triplet level T_{1A} of the acceptor molecule is larger than CT, non-radiative deactivation via the above-described route is sufficiently prevented, and it is considered that energy efficiency is improved.

Furthermore, in the OLED device of the present invention, since the excited triplet level T_{1D} of the donor molecule is smaller than the value obtained by adding 0.1 eV to CT, it is considered that the electron (−)/hole (+) pair can generate the triplet state of the donor molecule in the donor layer 2 through the CT state. Note that, although FIG. 3A describes an example in which light-emission derived from the donor molecule occurs, in a case where a dopant molecule is present in the donor layer 2, energy transfer from the donor molecule to the dopant molecule occurs, whereby light-emission derived from the dopant molecule occurs.

The excited triplet level T_{1D} of the donor molecule is preferably equal to or smaller than CT, from the viewpoint of further improving energy efficiency.

The donor layer preferably further contains a dopant molecule, from the viewpoint of further improving light-emission efficiency.

The donor layer may have a first layer forming an interface with the acceptor layer and a second layer laminated on the first layer, the first layer may contain a donor molecule that causes triplet-triplet annihilation, and the second layer may contain the donor molecule and a dopant molecule.

In addition, the donor layer may have a first layer forming an interface with the acceptor layer, a second layer laminated on the first layer, and a third layer laminated on the second layer, wherein the first layer contains a donor molecule that causes triplet-triplet annihilation, the second layer contains the donor molecule and a dopant molecule, and the third layer contains the donor molecule.

### Advantageous Effects of Invention

According to the present invention, an OLED device excellent in light-emission efficiency and luminance can be provided.

### BRIEF DESCRIPTION OF DRAWINGS

FIG. 1 is a conceptual diagram illustrating a mechanism in which light-emission is caused to occur by the OLED device of the present invention.

FIG. 2 is a view illustrating energy levels of rubrene, perylene, NDI-HF, and NDI-Cy used in Examples.

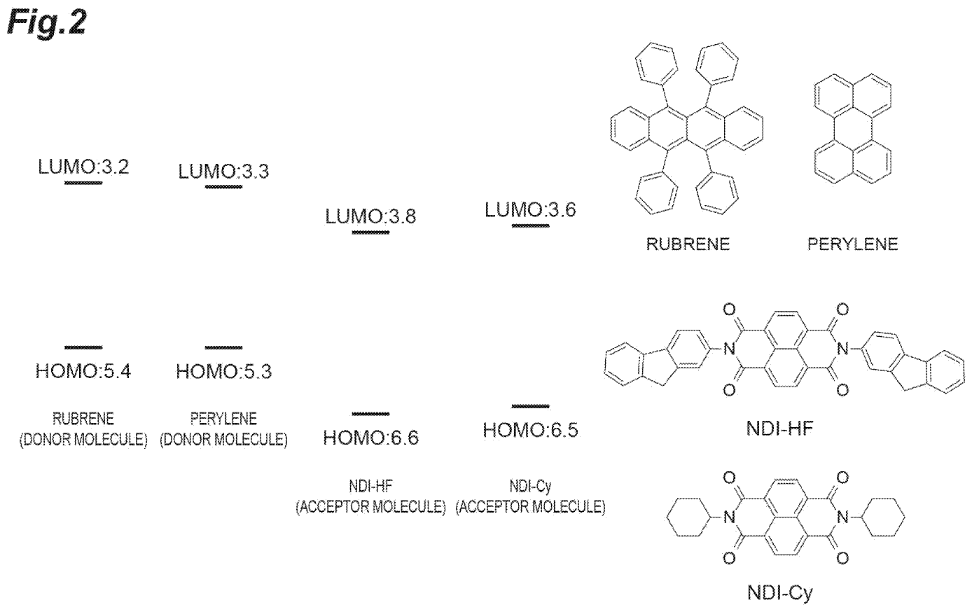

FIG. 3A is a schematic diagram illustrating an energy transfer mechanism leading to light-emission by the OLED device of the present invention, and FIG. 3B is a schematic diagram illustrating an energy transfer mechanism by an OLED device outside the scope of the present invention.

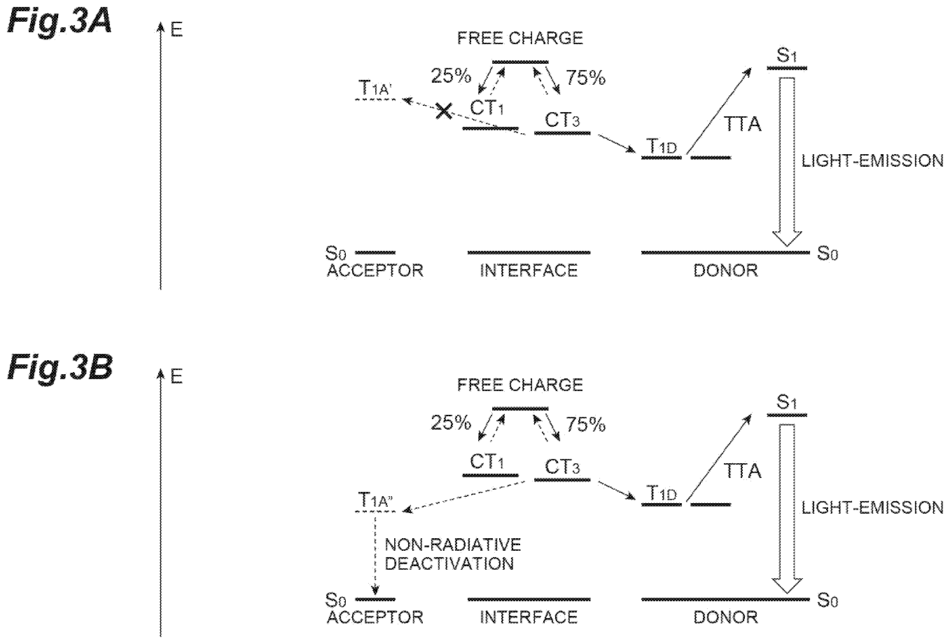

FIG. 4 is a view illustrating measurement results of the excited triplet level T₁ of NDI-HF.

FIG. 5 is a view illustrating measurement results of the excited triplet level T₁ of NDI-Cy.

FIG. 6 is a schematic cross-sectional view of the OLED device of Example 1.

FIG. 7 is a view illustrating measurement results of applied voltage-luminance characteristics of the OLED devices of Examples 1 and 2 and Comparative Example 1A.

FIG. 8 is a view illustrating measurement results of device lifetime of the OLED devices of Reference Example 1 and Comparative Example 1B.

FIG. 9 is a view illustrating measurement results of external quantum efficiency (EQE) of the OLED devices of Examples 1 and 3 and Reference Example 1.

FIG. 10 is a view illustrating measurement results of applied voltage-luminance characteristics of the OLED devices of Example 4 and Comparative Example 2.

### DESCRIPTION OF EMBODIMENTS

Hereinafter, one embodiment of the present invention will be described in detail. However, the present invention is not limited to the following embodiment.

The OLED device of the present embodiment includes a layered structure sandwiched between a pair of electrodes.

The layered structure has an acceptor layer containing an acceptor molecule and a donor layer containing a donor molecule, and the acceptor layer and the donor layer form an interface. The layered structure may have an organic semiconductor layer other than the acceptor layer and the donor layer, and may further include an inorganic compound layer such as a molybdenum trioxide (MoO₃) layer (hole transport layer or electron injection layer) or a lithium fluoride layer (electron injection layer) between the electrodes.

The acceptor layer may be formed only of the acceptor molecule, or may contain a material other than the acceptor molecule within a range that does not significantly impair the effect of the present invention.

The donor molecule is a material that causes triplet-triplet annihilation. The donor layer may be formed only of the donor molecule, may be formed only of the donor molecule and a dopant molecule, or may contain a material other than the donor molecule and the dopant molecule within a range that does not significantly impair the effect of the present invention.

The donor layer may be formed of a plurality of layers. For example, the donor layer may be a two-layer donor layer having a first layer forming an interface with the acceptor layer and a second layer laminated on the first layer (a second layer facing the acceptor layer with the first layer interposed therebetween), or may be a three-layer donor layer having a first layer forming an interface with the acceptor layer, a second layer laminated on the first layer (a second layer facing the acceptor layer with the first layer interposed therebetween), and a third layer laminated on the second layer (a third layer facing the first layer with the second layer interposed therebetween). Note that, when the donor layer is formed of a plurality of layers, each layer contains the same donor molecule.

In a case where the donor layer contains a donor molecule and a dopant molecule, the donor layer preferably has an intermediate layer (hereinafter also referred to as “intermediate layer”) that contains the donor molecule but does not contain the dopant molecule, between the acceptor layer and the layer containing the donor molecule and the dopant molecule, from the viewpoint of further improving light-emission efficiency. The reason why light-emission efficiency is improved by the donor layer having the intermediate layer is not necessarily clear, but it is considered that one reason is that excitonic interface quenching can be prevented because light-emission occurs away from the interface between the donor layer and the acceptor layer.

The thickness of the intermediate layer, if present, can be, for example, 1 to 10 nm, and preferably 1 to 5 nm.

The above-described two-layer donor layer can be configured, for example, such that the first layer serves as the above-described intermediate layer and the second layer is composed of the donor molecule and the dopant molecule.

The above-described three-layer donor layer can be configured, for example, such that the first layer serves as the above-described intermediate layer, the second layer is composed of the donor molecule and the dopant molecule, and the third layer is composed of the donor molecule.

The HOMO level of the acceptor molecule is lower than the HOMO level of the donor molecule. The difference between the HOMO level of the acceptor molecule and the HOMO level of the donor molecule is preferably 0.5 eV or more, from the viewpoint of highly preventing hole leakage to further improve light-emission efficiency. Note that the upper limit of the difference between the HOMO level of the acceptor molecule and the HOMO level of the donor molecule is not particularly limited, but can be set to, for example, 2 eV or less.

The LUMO level of the acceptor molecule is lower than the LUMO level of the donor molecule. The difference between the LUMO level of the acceptor molecule and the LUMO level of the donor molecule is preferably 0.3 eV or more, from the viewpoint of highly preventing electron leakage to further improve light-emission efficiency. Note that the upper limit of the difference between the LUMO level of the acceptor molecule and the LUMO level of the donor molecule is not particularly limited, but can be set to, for example, 2 eV or less.

The excited triplet level T_{1D} of the donor molecule is smaller than the value obtained by adding 0.1 eV to CT, and is preferably equal to or smaller than CT. The difference between the excited triplet level T_{1D} of the donor molecule and CT is preferably less than 0.8 eV, more preferably less than 0.7 eV, and still more preferably less than 0.6 eV. When this difference is small, the light-emission initiation voltage can be lowered.

CT is smaller than the energy difference between the HOMO level and the LUMO level of the donor molecule by 0.4 eV or more. The upper limit of CT is not particularly limited, but can be set to, for example, 2 eV or less.

In a case where the donor layer contains a donor molecule and a dopant molecule, the energy difference between the HOMO level and the LUMO level of the dopant molecule is preferably smaller than the energy difference between the HOMO level and the LUMO level of the donor molecule. When the dopant molecule is used, energy transfer from the donor molecule to the dopant molecule occurs, and light-emission derived from the dopant molecule can be realized.

The content of the dopant molecule in the donor layer can be set to, for example, 0.01 to 50% by volume, and preferably 0.1 to 10% by volume, with respect to a total of 100% by volume of the donor layer.

As the acceptor molecule, for example, a known electron transport material in the related art can be employed. Specific examples thereof include the following compounds.

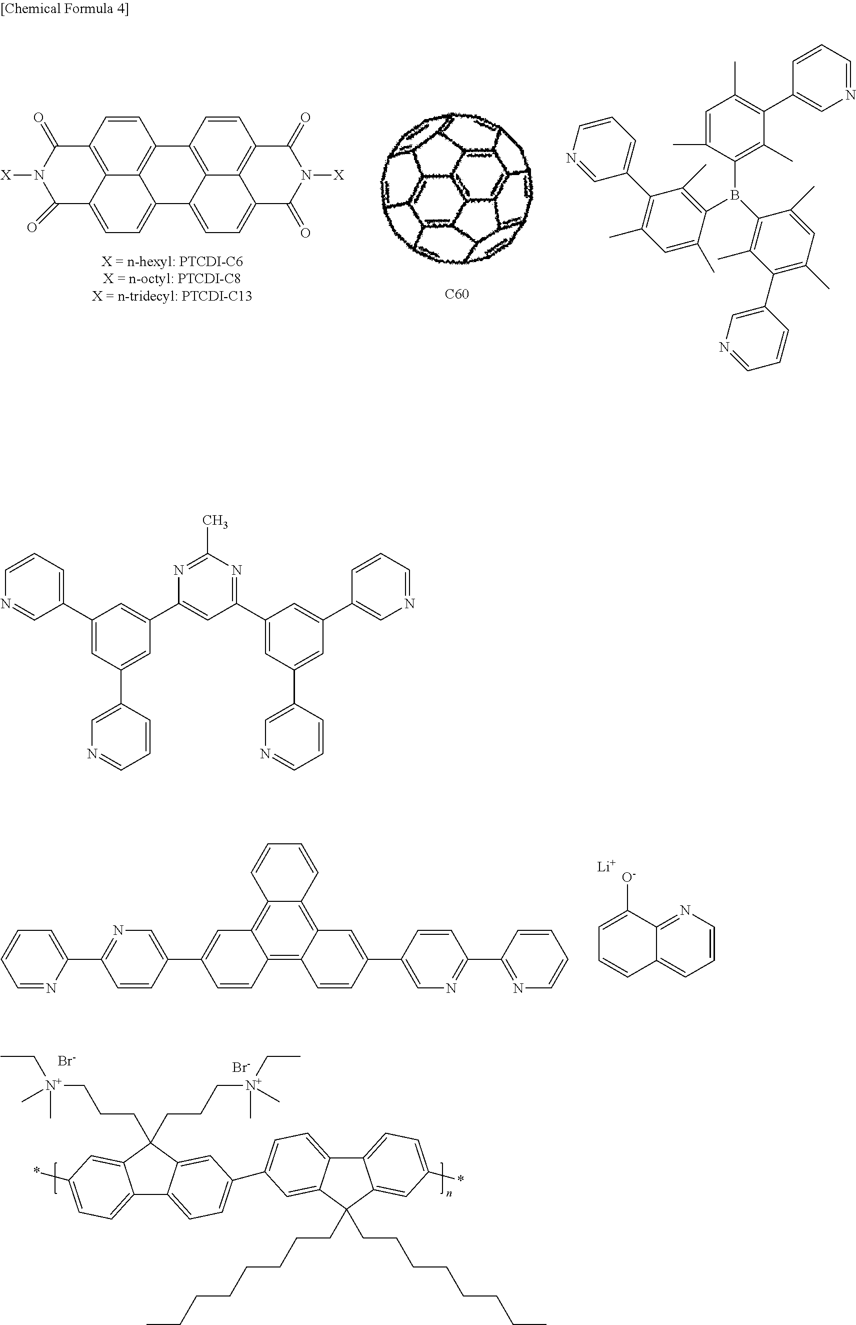

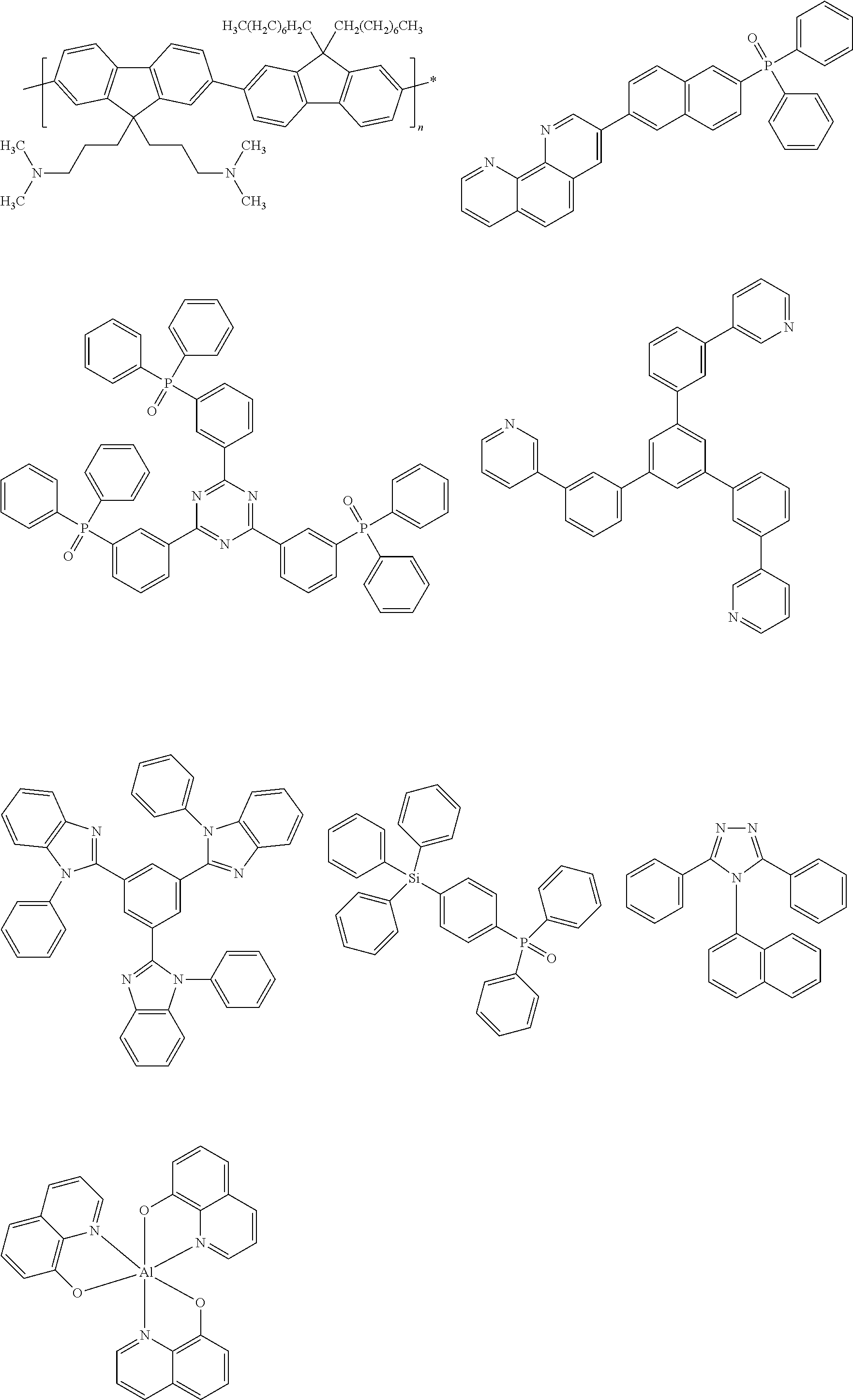

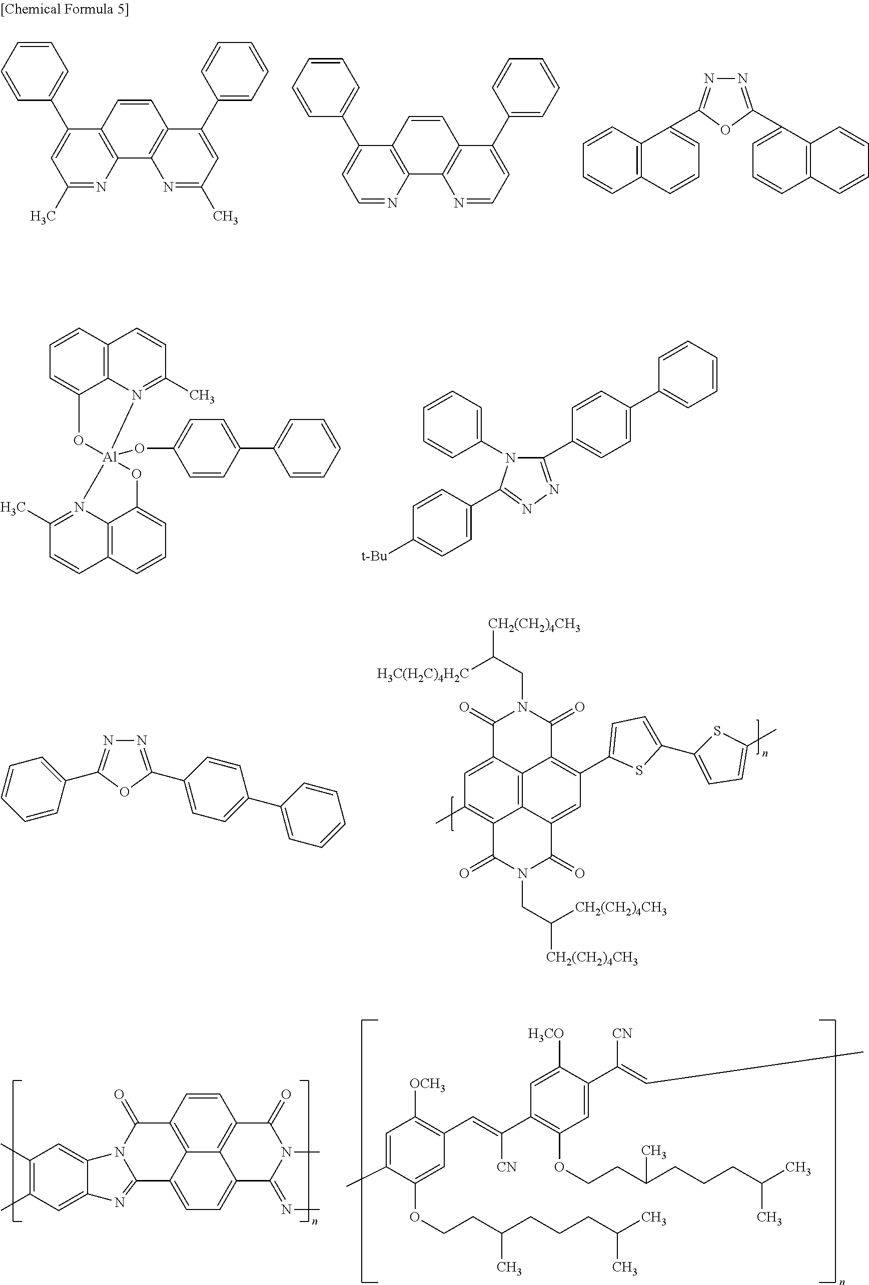

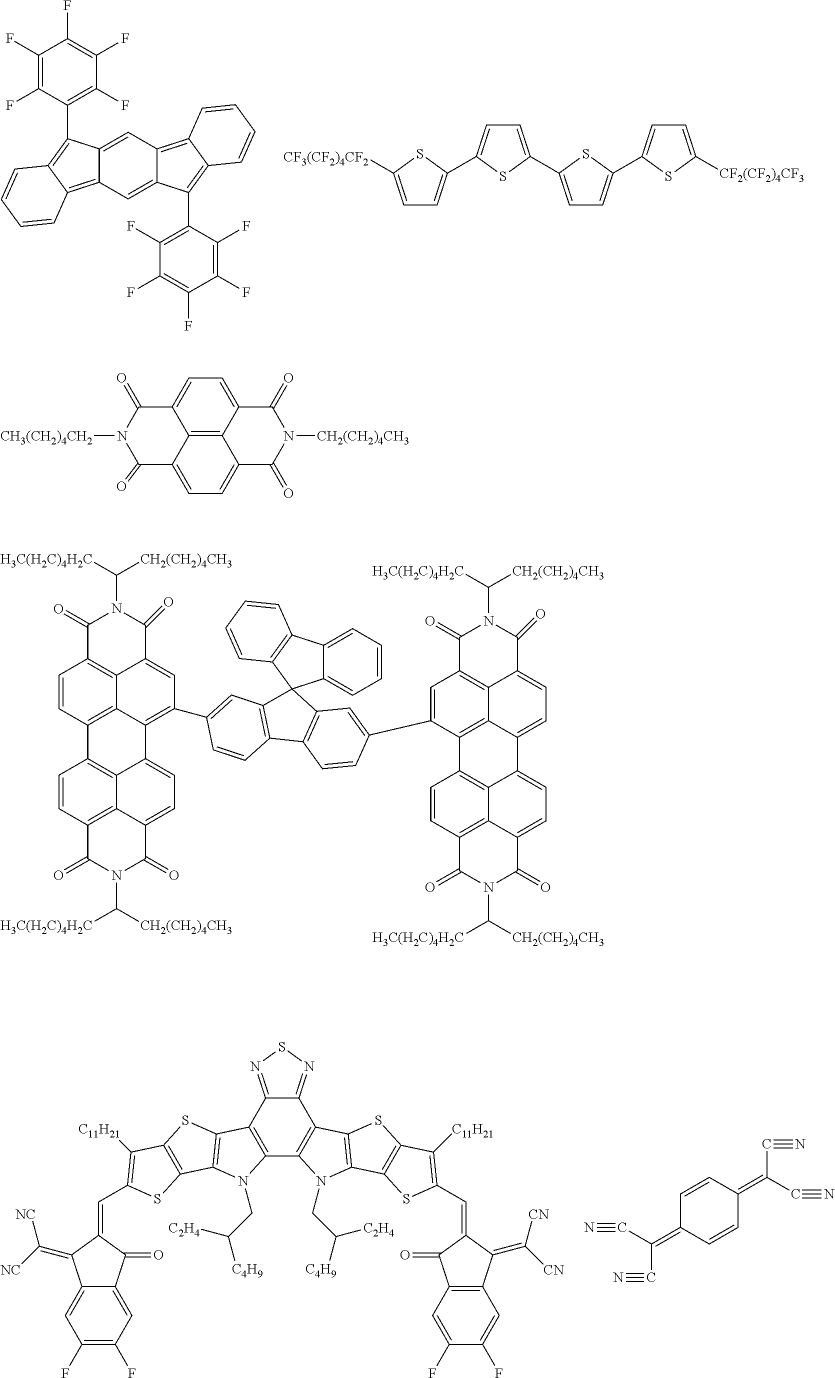

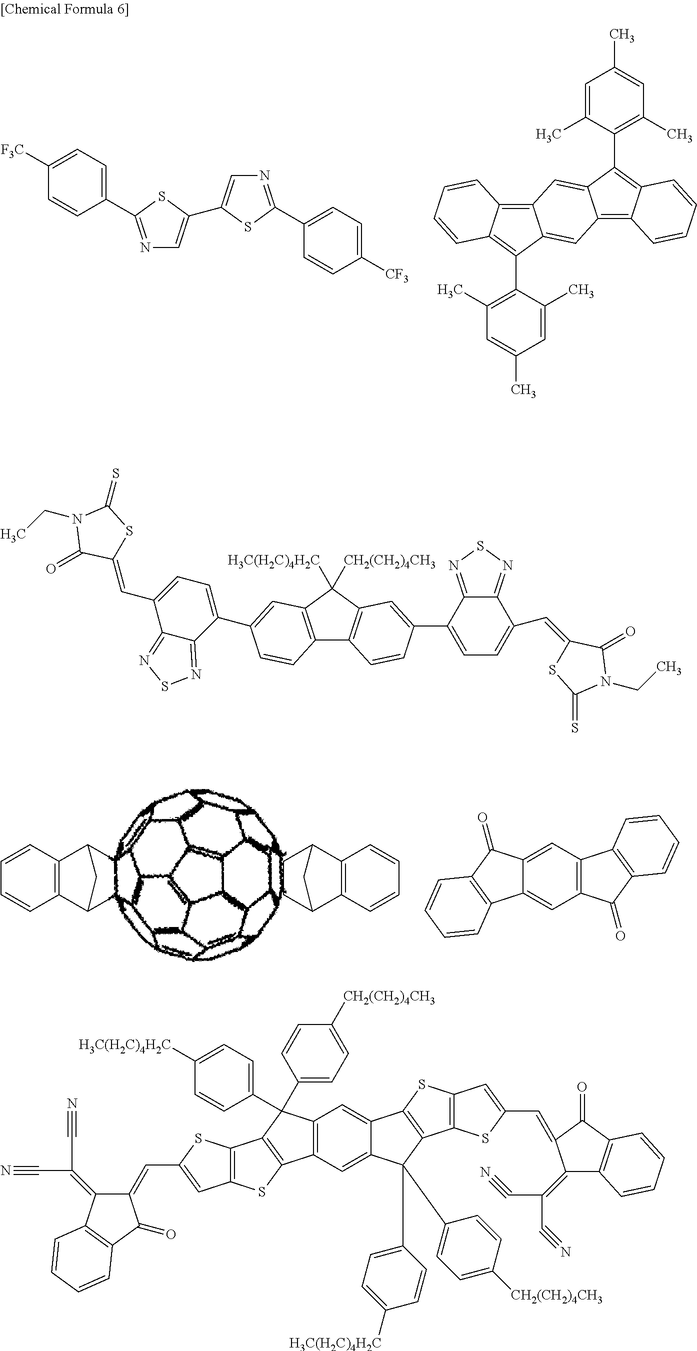

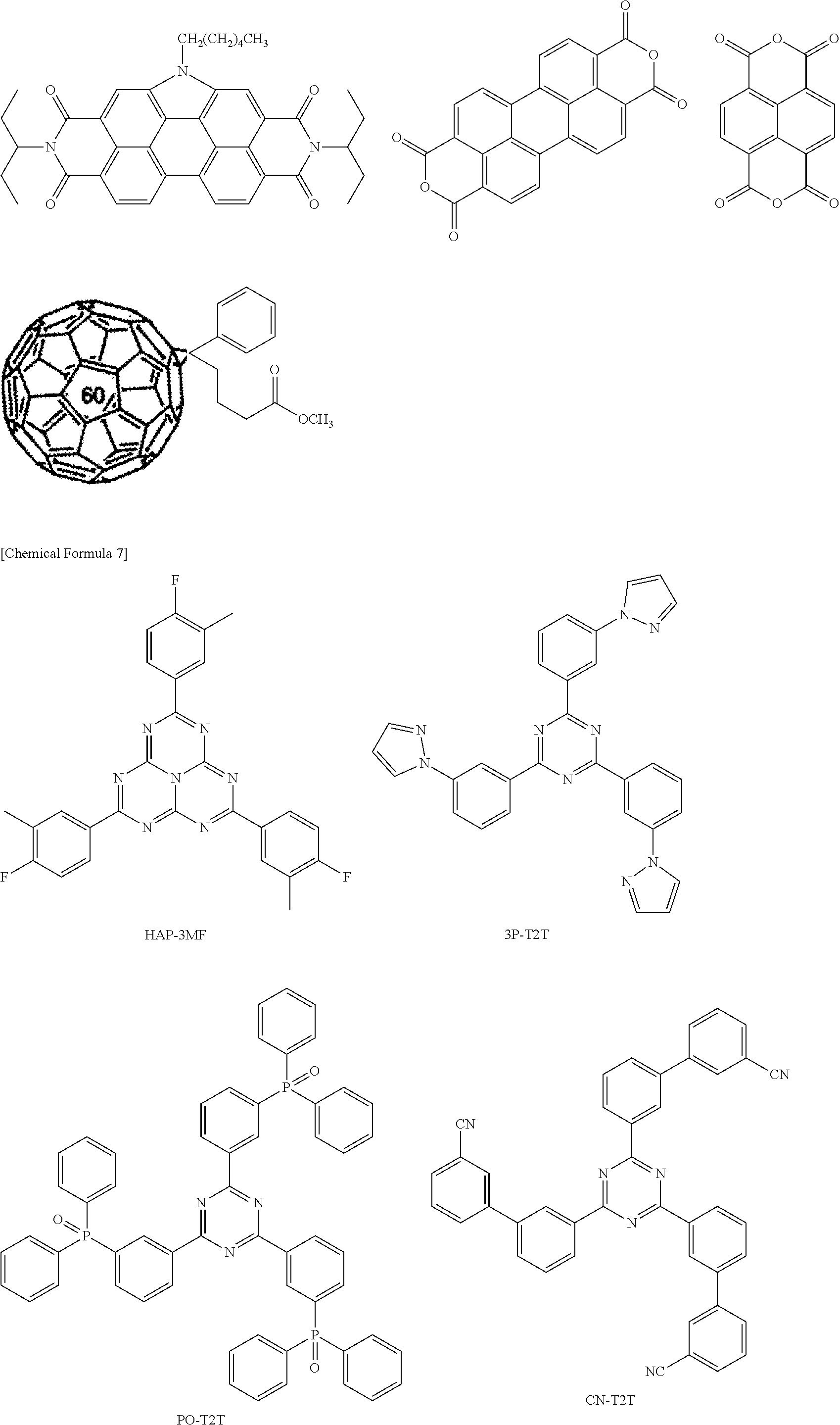

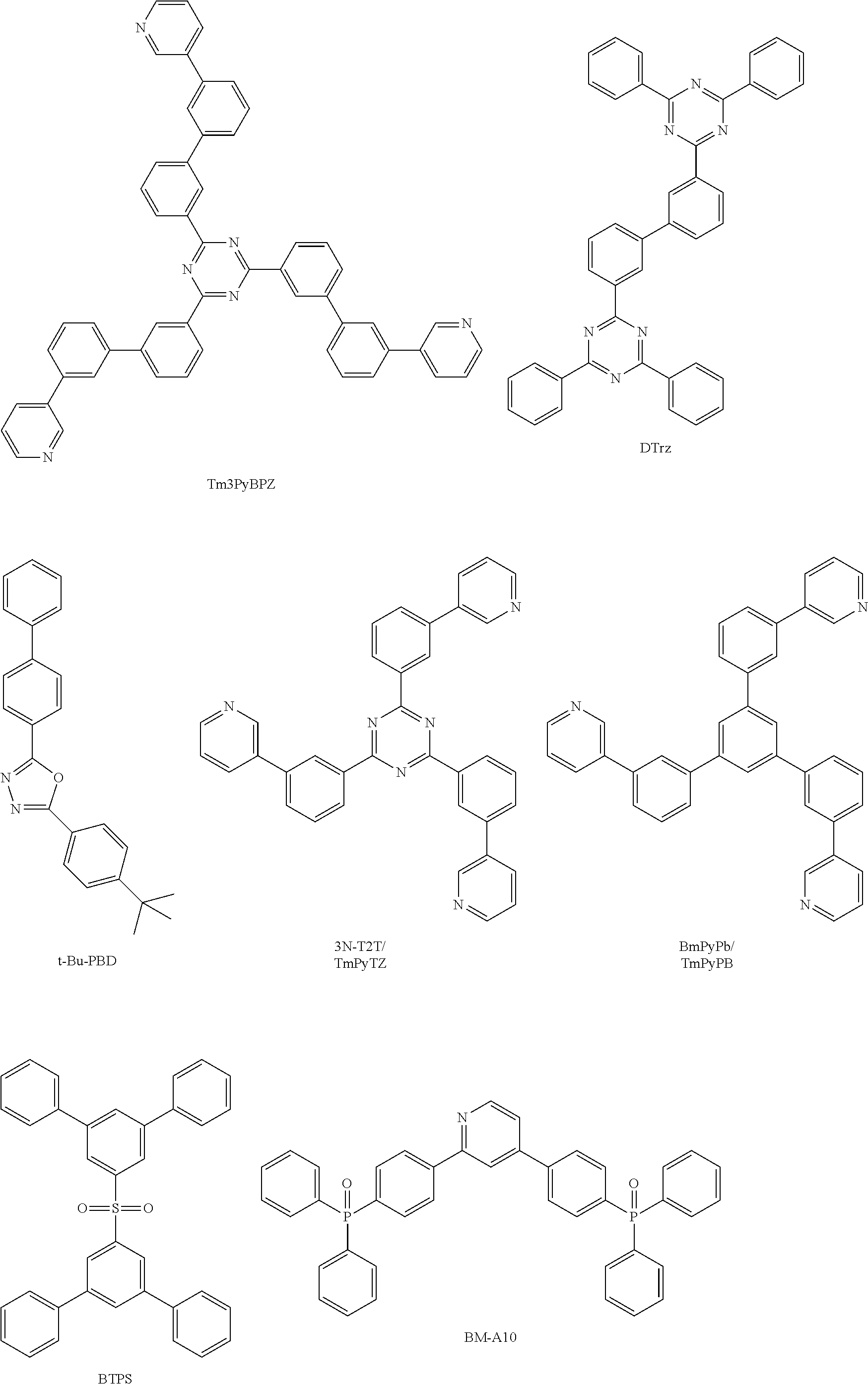

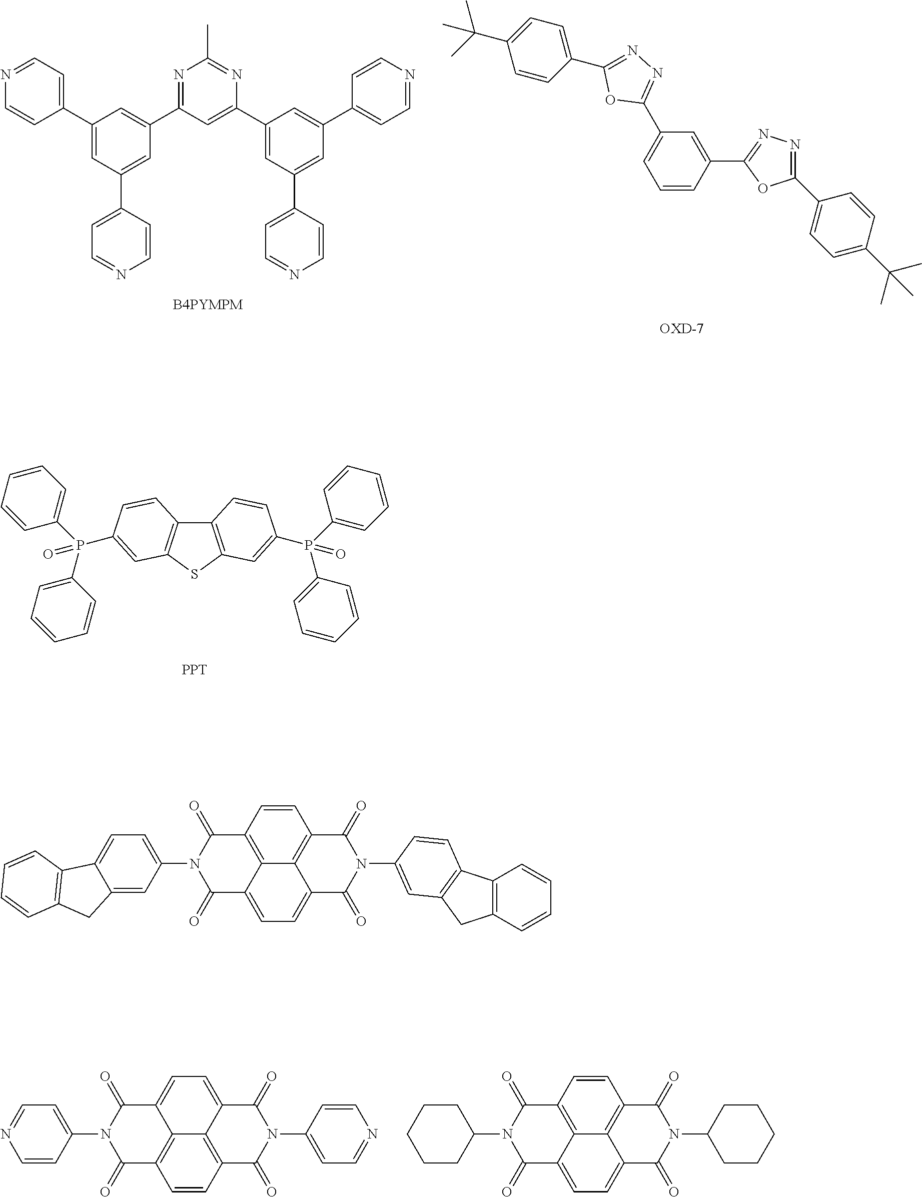

As the donor molecule, for example, the following compounds which are reported to cause TTA to occur can be employed.

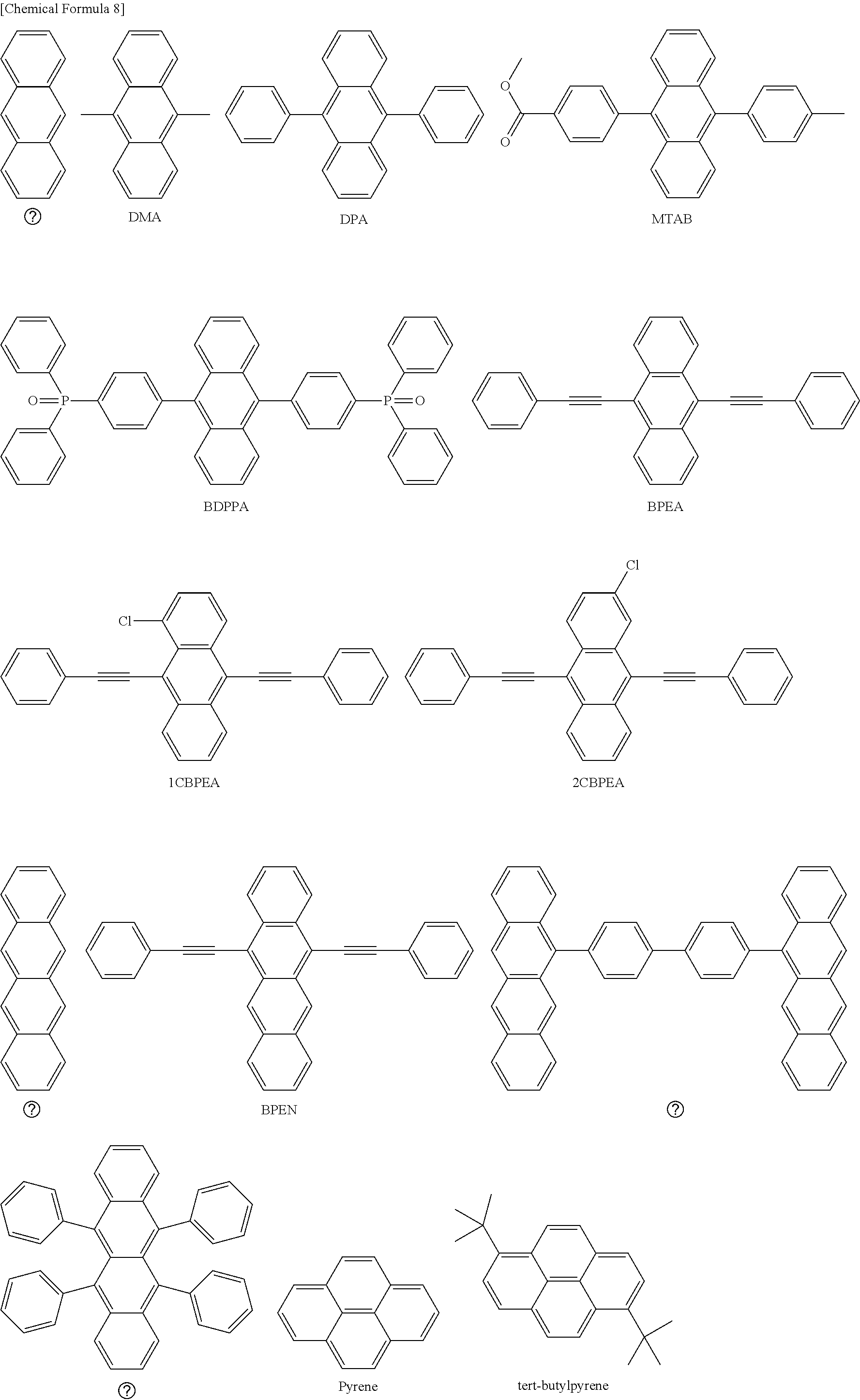

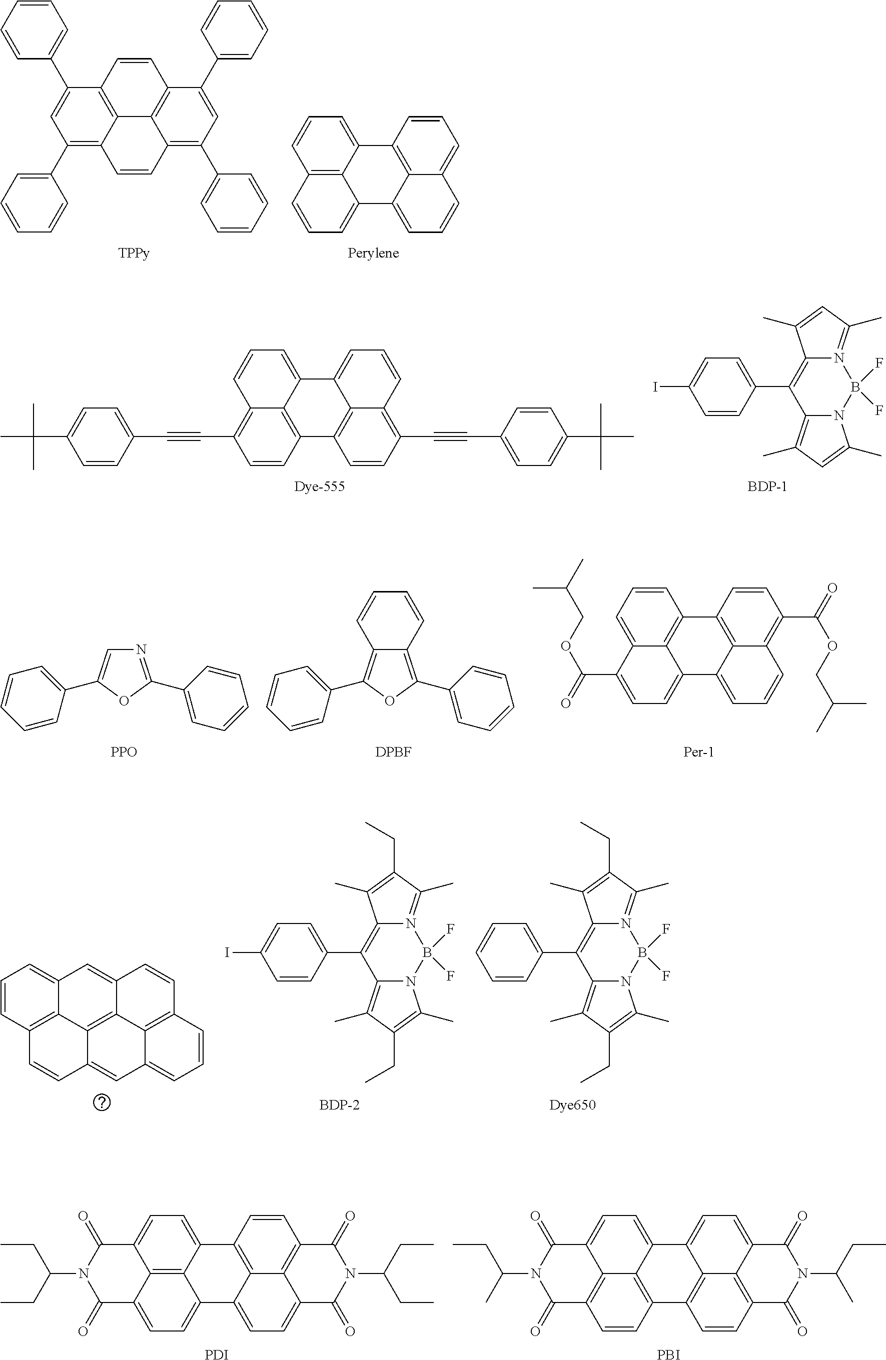

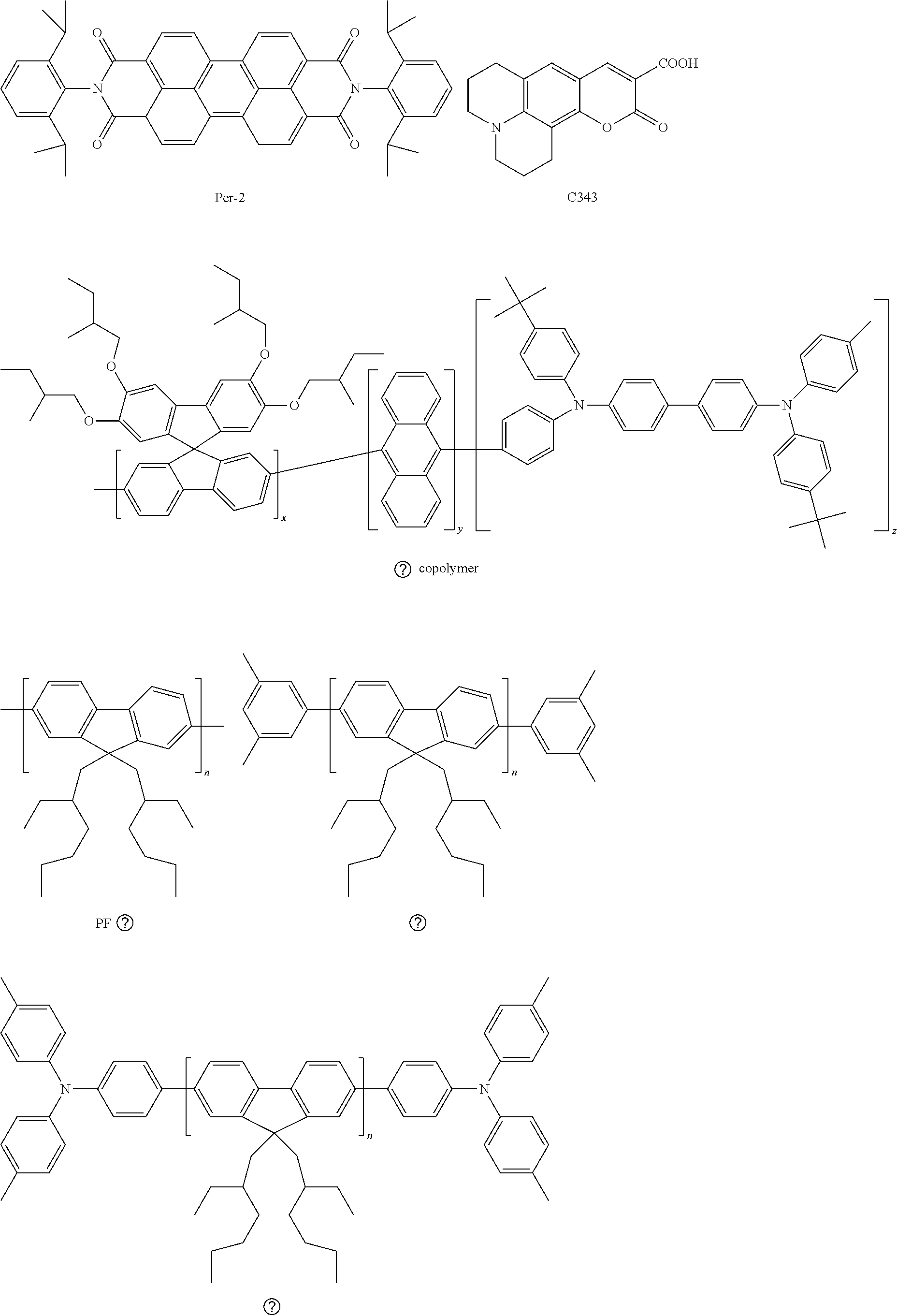

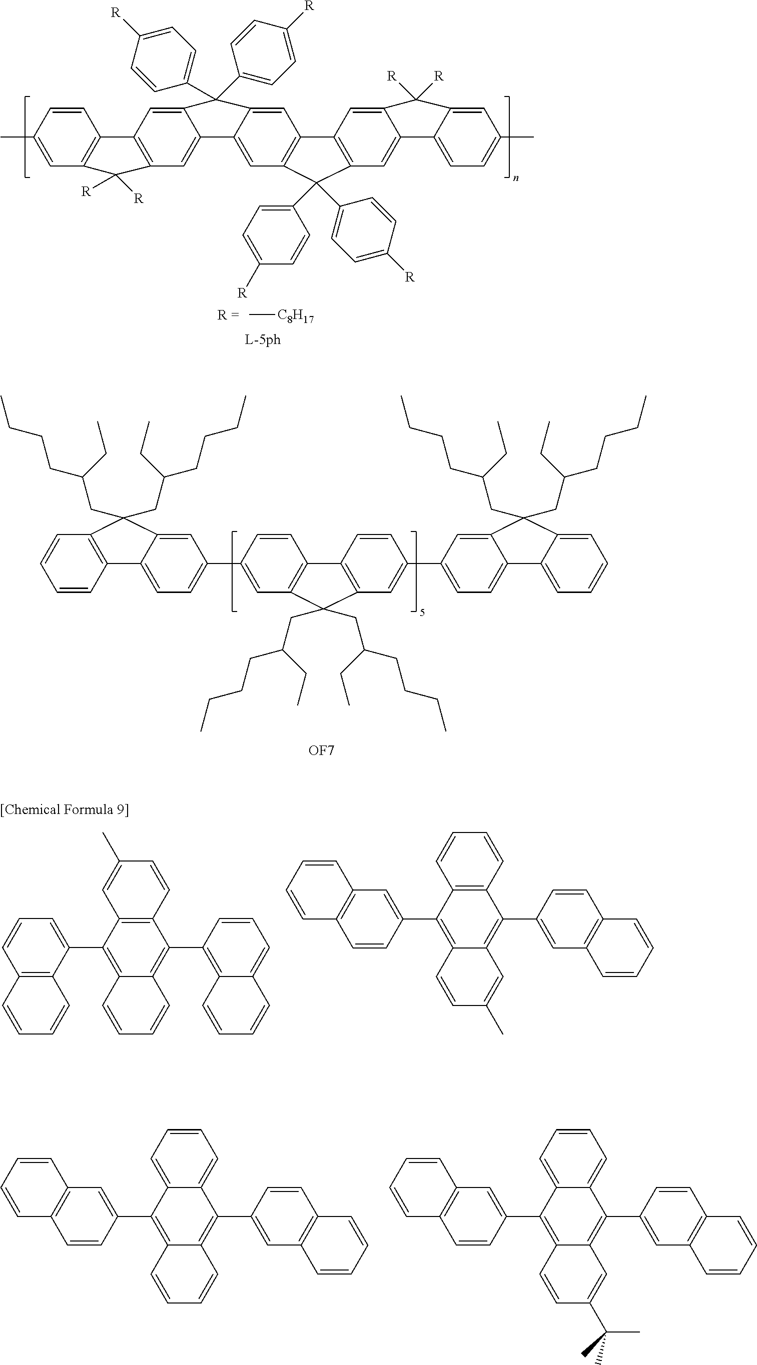

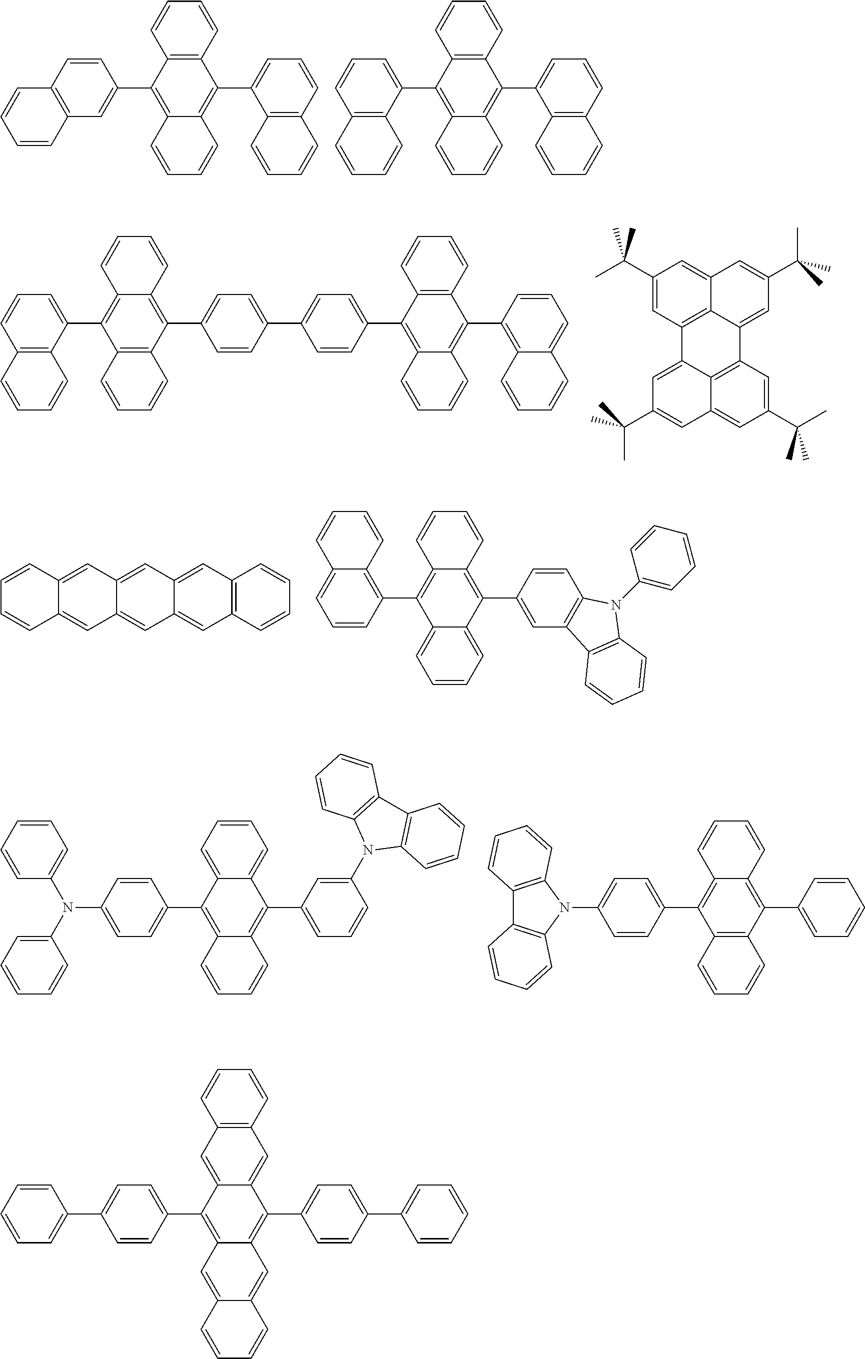

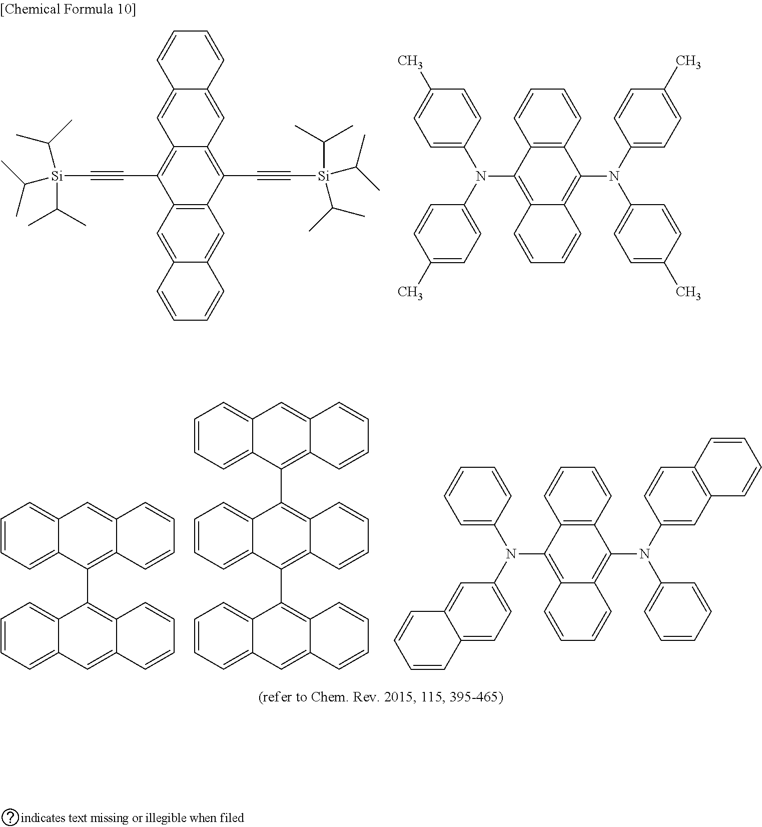

Energy levels such as a HOMO level, a LUMO level, and an excited triplet level T₁ of these compounds are inherent to the materials, and literature values and the like can be referred to.

As the dopant molecule, a known light-emitting material in the related art can be employed. Specific examples of the dopant molecule include the following compounds.

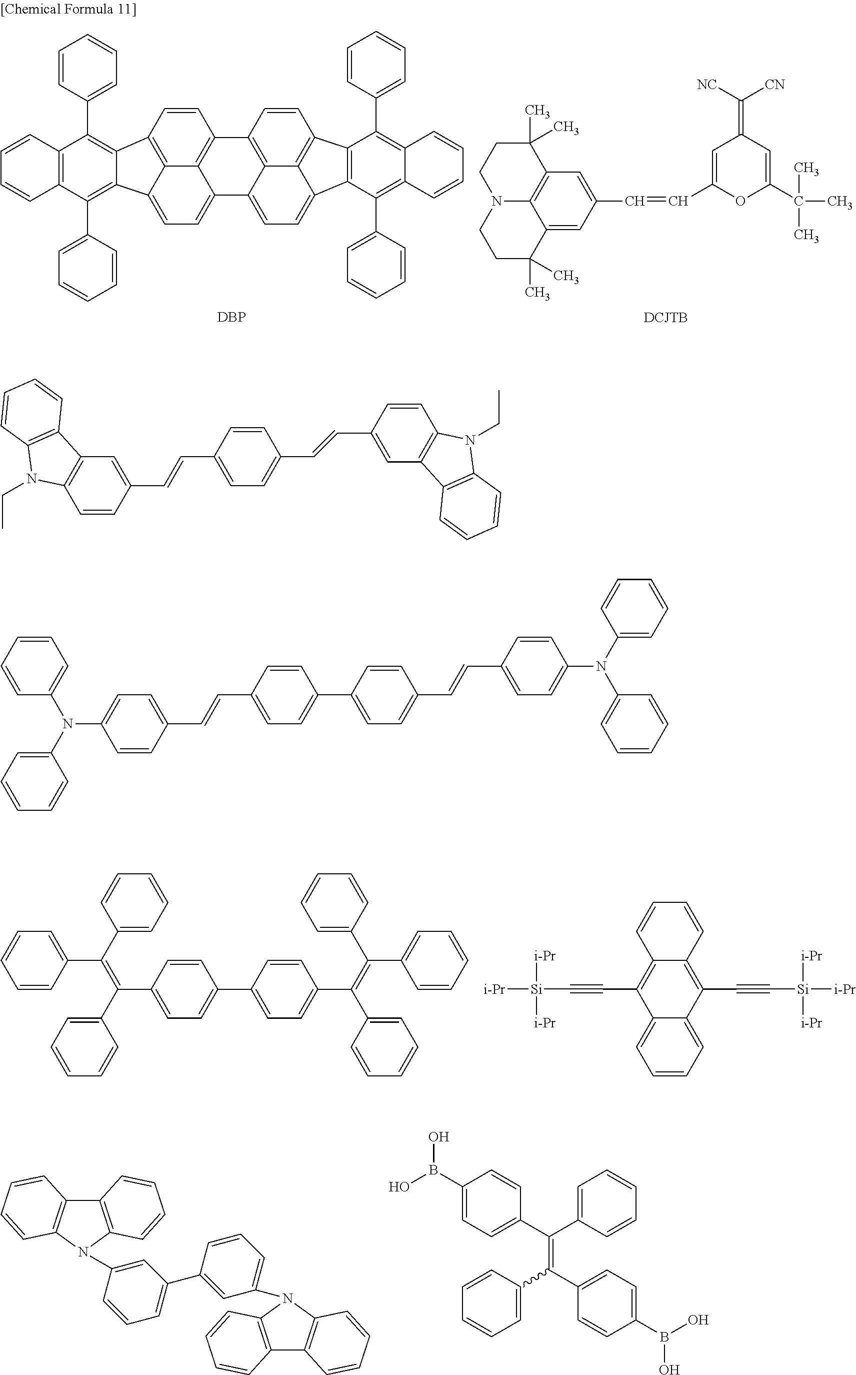

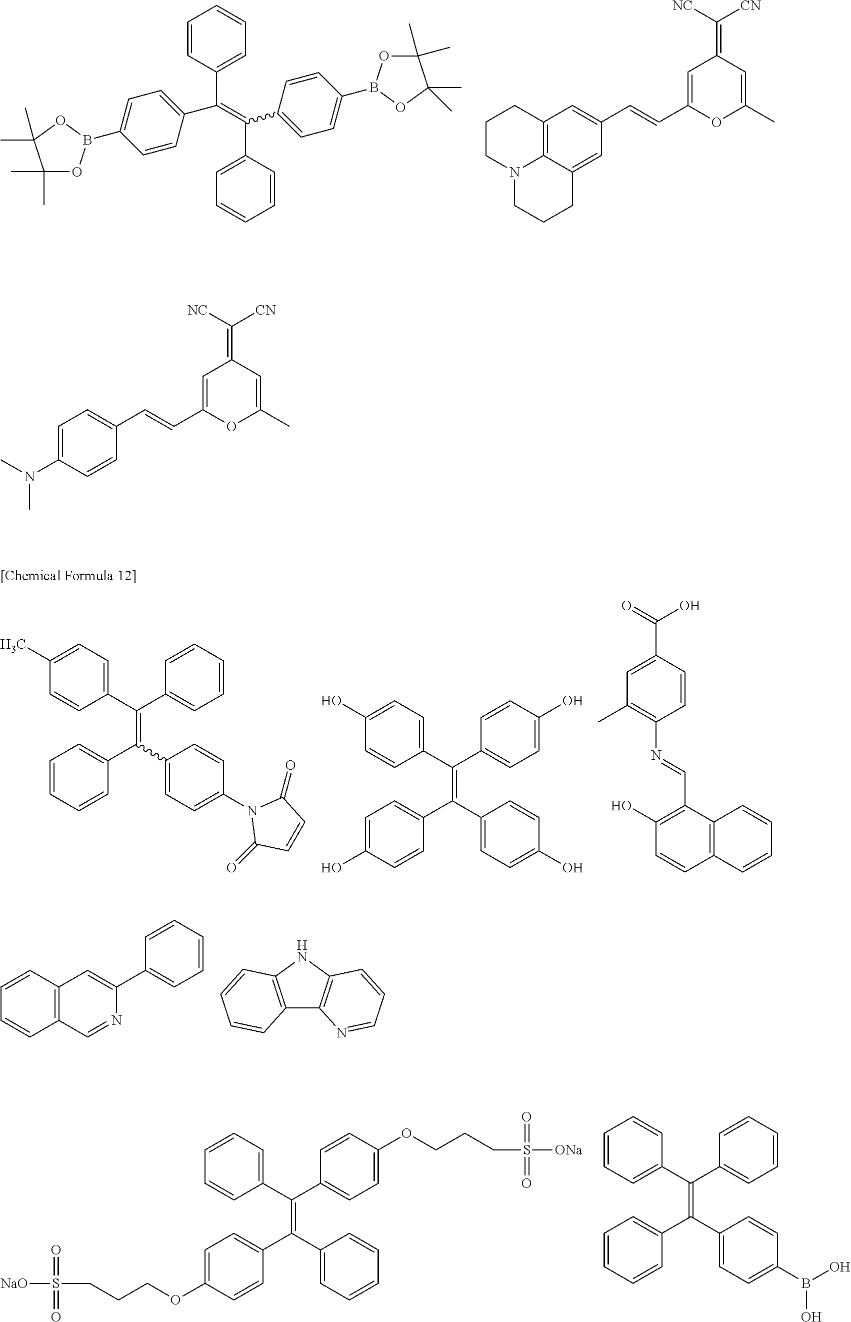

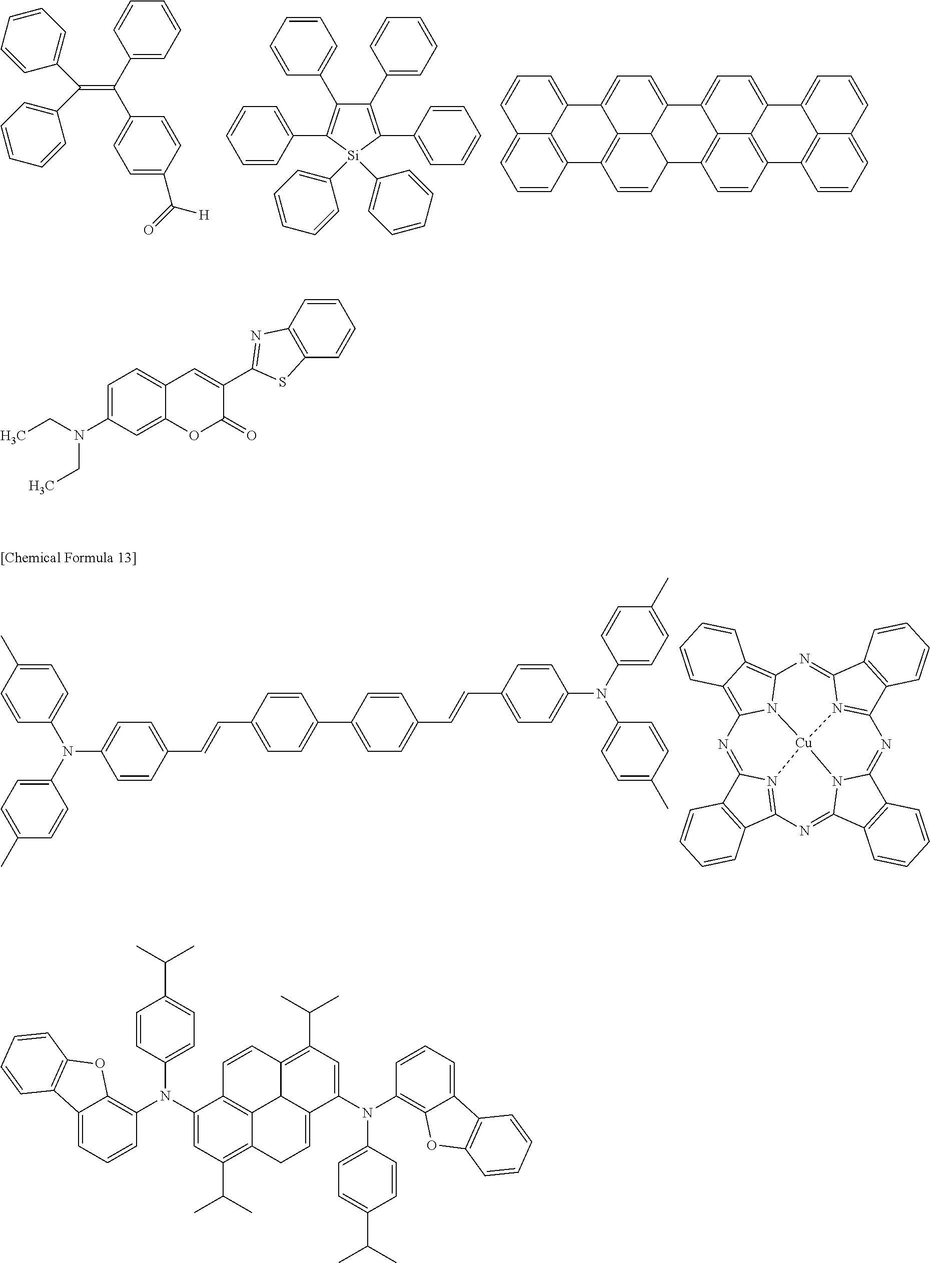

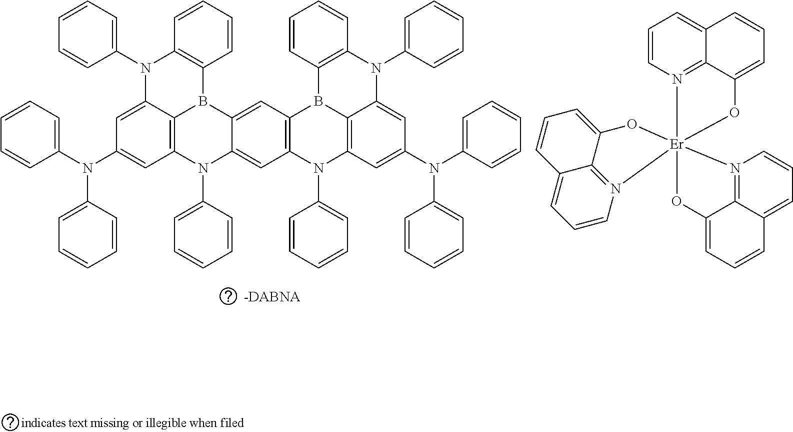

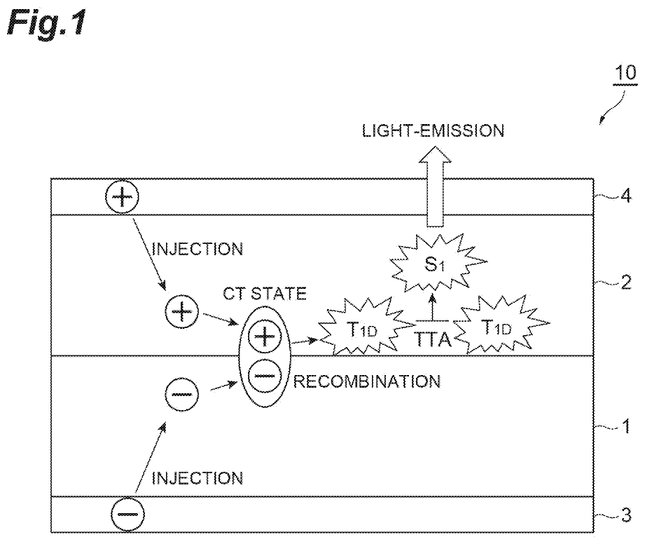

As illustrated in FIG. 1, the OLED device of the present embodiment may be formed of a pair of electrodes, the acceptor layer, and the donor layer, or may include other organic semiconductor layers, other inorganic compound layers, and the like which are known in the related art.

Examples of layers which may be provided between the pair of electrodes in the OLED device of the present embodiment include, in this order from an anode, a hole injection layer, an electron block layer, a hole transport layer, a light-emitting layer, an electron transport layer, a hole block layer, and an electron injection layer. Among these, the acceptor layer may be the electron transport layer, and the donor layer may be the light-emitting layer.

Note that functions of these layers are not strictly distinguished. For example, the light-emitting layer that is the donor layer may also function as the hole transport layer, or the hole block layer may also function as the electron injection layer.

The order of the respective layers in the OLED device is also not limited to the above-described order, and for example, the hole injection layer may be located between the electron block layer and the hole transport layer, and the electron injection layer may be located between the hole block layer and the electron transport layer.

Among the respective layers of the OLED device, there may be layers containing the same organic semiconductor material. For example, in a case where the OLED device contains rubrene as the donor molecule in the donor layer (light-emitting layer), the OLED device may have a hole block layer formed of rubrene.

The OLED device of the present embodiment can be manufactured by forming the acceptor layer and the donor layer by a known method in the related art, for example, a method such as a vacuum evaporation method, a chemical vapor deposition method, a sputtering method, a vapor deposition polymerization method, a spin coating method, a blade coating method, a bar coating method, a dip coating method, or a laminating method. Specifically, for example, the OLED device of the present embodiment can be manufactured by stacking the first electrode, the acceptor layer, the donor layer, the second electrode, and any other layers on a substrate. A method of forming the respective organic semiconductor layers can be appropriately selected depending on the compound. As the substrate, for example, a glass substrate, a quartz substrate, a sapphire substrate, a plastic substrate, a film substrate, or the like can be employed.

The thicknesses of the acceptor layer and the donor layer in the OLED device of the present embodiment are not particularly limited, but are preferably 0.1 nm to 500 nm, and more preferably 2 nm to 200 nm.

The OLED device is expected to be applied to, for example, an organic EL display, organic EL lighting, digital signage, a light source for a photosensor, a laser light source, a light source for optical communication, and the like.

### EXAMPLES

Hereinafter, the present invention will be described in more detail with reference to Examples, but the present invention is not limited to Examples at all. Note that structures of compounds used in Examples are shown below.

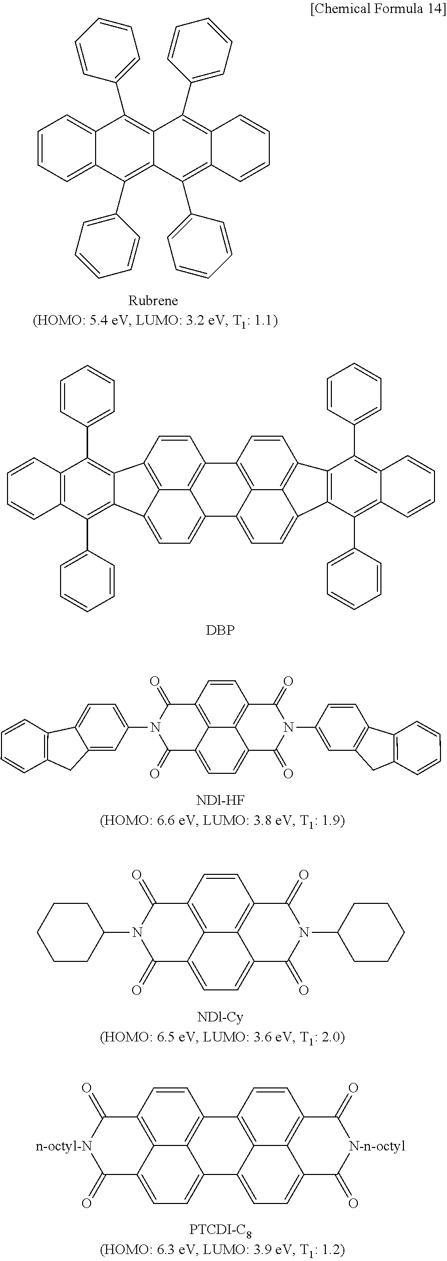

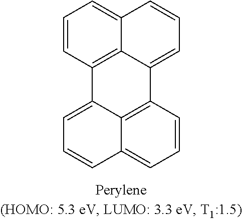 [SMILES: c1cc2cccc3c4cccc5cccc(c(c1)c23)c54]

### (Measurement of Excited Triplet Level T₁ of NDI-HF and NDI-Cy)

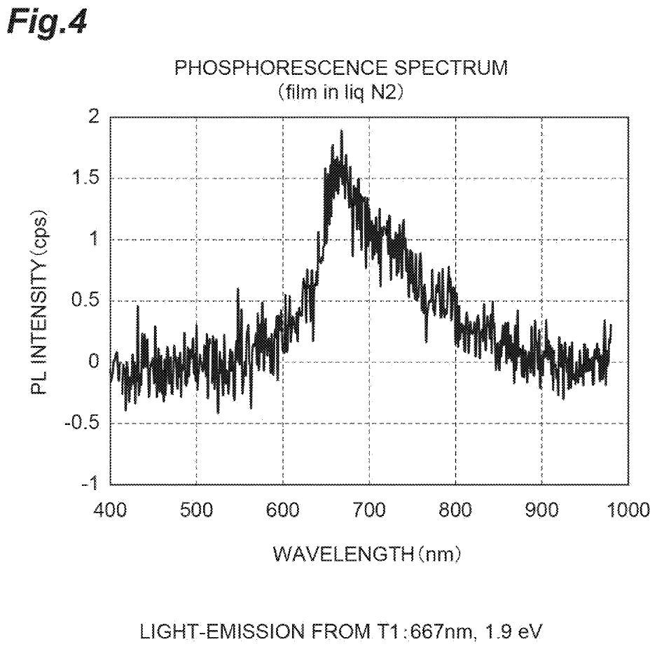

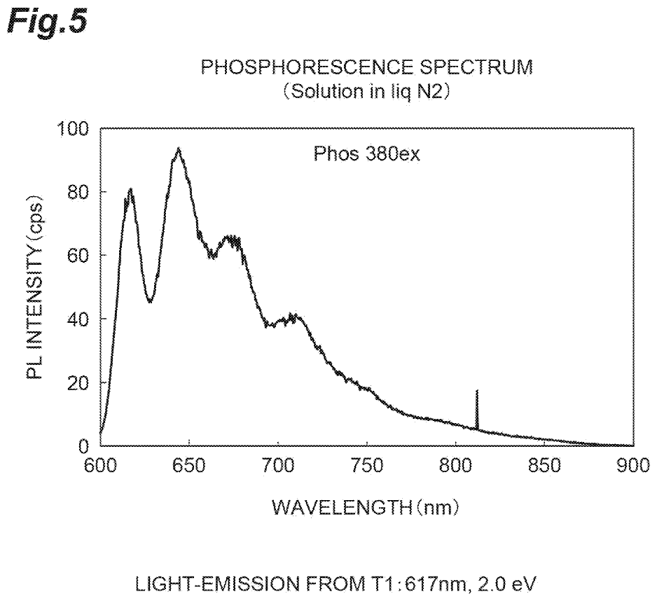

The excited triplet level T₁ was determined from the peak intensity by measuring a phosphorescence spectrum at liquid nitrogen temperature using a spectrofluorophotometer (model FP-8650, manufactured by JASCO Corporation). When a plurality of peaks were detected, the peak on the higher energy side was adopted. NDI-HF was used as a sample by depositing a thin film having a thickness of 50 nm on a quartz substrate, and NDI-Cy was used as a sample by placing a chloroform solution at a concentration of 10⁻⁵ mol/L into a sample tube having a diameter of 5 mm. The measurement results of the excited triplet level T₁ for NDI-HF and NDI-Cy are shown in FIG. 4 and FIG. 5, respectively.

### Example 1

An MoO₃ hole transport layer (10 nm), a rubrene layer (non-doped) (10 nm), a rubrene layer (doped with DBP) (100 nm), a rubrene layer (non-doped) (2 nm), an NDI-HF layer (50 nm), an LiF electron injection layer (3 nm), an MoO₃ electron injection layer (3 nm), and an Al electrode (100 nm) were thermally evaporated in this order in a high-vacuum state (about 10⁻⁵ Pa) in a vacuum evaporation system onto a glass substrate coated with indium tin oxide (ITO) (the thickness of ITO: 150 nm, a sheet resistance: 10.3 Ω/sq., manufactured by Techno Print Co., Ltd.). The resultant device was sealed with a glass substrate and an epoxy resin in a glove box to obtain an OLED device. Note that the rubrene layer (doped with DBP) was formed by adding DBP as a dopant in an amount of 0.5% by volume with respect to the entirety of the rubrene layer and introducing DBP into the rubrene layer by a co-evaporation method. Note that the mixing concentration was controlled by a ratio of evaporation rates.

The obtained OLED device has the following configuration.
ITO electrode/MoO₃ hole transport layer/rubrene layer (non-doped)/rubrene layer (doped with DBP)/rubrene layer (non-doped)/NDI-HF layer/LiF electron injection layer/MoO₃ electron injection layer/Al electrode

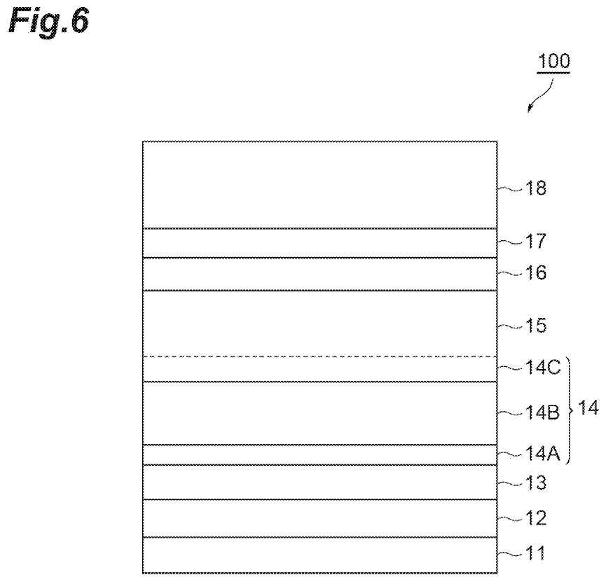

FIG. 6 is a schematic cross-sectional view of the OLED device of Example 1. The OLED device 100 illustrated in FIG. 6 has a structure in which a glass substrate 11, an ITO electrode 12, an MoO₃ hole transport layer 13, a rubrene (non-doped) layer 14A, a rubrene (doped with DBP) layer 14B, a rubrene (non-doped) layer 14C, an NDI-HF layer 15, an LiF electron injection layer 16, an MoO₃ electron injection layer 17, and an Al electrode 18 are laminated in this order.

Note that, in the OLED device 100, the rubrene (non-doped) layer 14A, the rubrene (doped with DBP) layer 14B, and the rubrene (non-doped) layer 14C correspond to the donor layer 14, and the NDI-HF layer 15 corresponds to the acceptor layer.

### Example 2

An OLED device was prepared in a similar manner as in Example 1 except that an NDI-Cy layer (50 nm) was formed by thermal evaporation instead of the NDI-HF layer. The OLED device has the following configuration.
ITO electrode/MoO₃ hole transport layer/rubrene layer (non-doped)/rubrene layer (doped with DBP)/rubrene layer (non-doped)/NDI-Cy layer/LiF electron injection layer/MoO₃ electron injection layer/Al electrode

### Comparative Example 1A

An OLED device was prepared in a similar manner as in Example 1 except that a PTCDI-C₈ layer (50 nm) was formed by thermal evaporation instead of the NDI-HF layer. The OLED device has the following configuration.
ITO electrode/MoO₃ hole transport layer/rubrene layer (non-doped)/rubrene layer (doped with DBP)/rubrene layer (non-doped)/PTCDI-C8 layer/LiF electron injection layer/MoO₃ electron injection layer/Al electrode

### Comparative Example 1B

An OLED device was prepared in a similar manner as in Comparative Example 1A except that the rubrene layer (non-doped) in contact with the PTCDI-C₈ layer was excluded. The OLED device has the following configuration.
ITO electrode/MoO₃ hole transport layer/rubrene layer (non-doped)/rubrene layer (doped with DBP)/PTCDI-C8 layer/LiF electron injection layer/MoO₃ electron injection layer/Al electrode

### <Evaluation 1 of OLED Device>

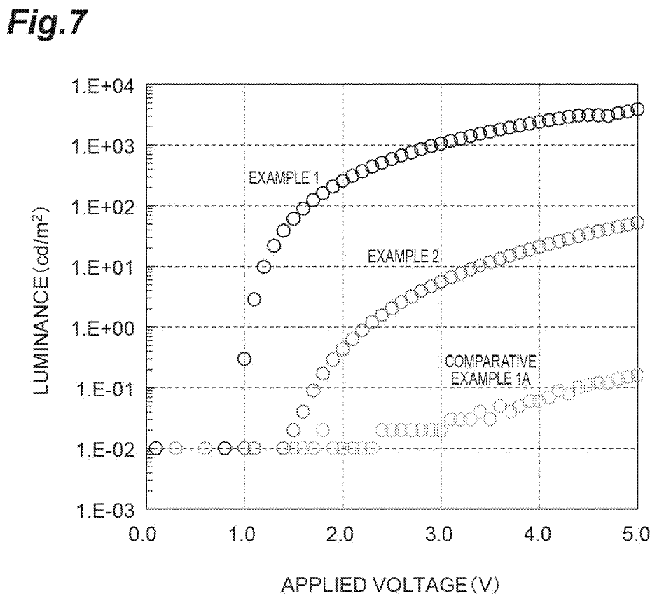

The applied voltage-luminance characteristics of the OLED devices of Examples 1 and 2 and Comparative Example 1A were measured using a source measure unit (B2902A, manufactured by Keysight Technologies) and a luminance meter (BM-9, manufactured by TOPCOM CORPORATION). The results are shown in FIG. 7.

As is clear from FIG. 7, the OLED devices of Examples 1 and 2 have excellent luminance in comparison to the OLED device of Comparative Example 1A.

### Example 3: Examination of Film Thickness of Intermediate Layer

An OLED device was prepared in a similar manner as in Example 1 except that the film thickness of the intermediate layer (rubrene layer (non-doped)) corresponding to the first layer of the donor layer was changed from 2 nm to 4 nm.

### Reference Example 1: Examination of Case where Intermediate Layer is Absent

An OLED device was prepared in a similar manner as in Example 1 except that the intermediate layer (rubrene layer (non-doped)) corresponding to the first layer of the donor layer was excluded.

The obtained OLED device has the following configuration.
ITO electrode/MoO₃ hole transport layer/rubrene layer (non-doped)/rubrene layer (doped with DBP)/NDI-HF layer/LiF electron injection layer/MoO₃ electron injection layer/Al electrode

### <Evaluation of Lifetime>

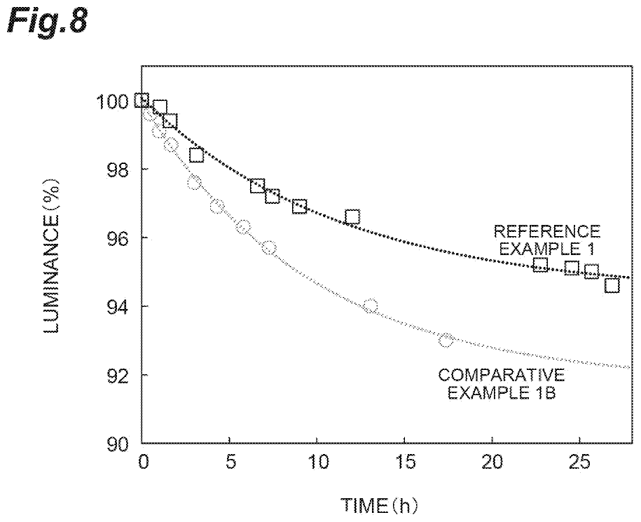

The device lifetime of the OLED devices of Reference Example 1 and Comparative Example 1B (evaluated by LT95, which is the time until the luminance decreases to 95% with the initial luminance taken as 100%) is shown in FIG. 8. As is clear from FIG. 8, the OLED device of Reference Example 1 has an LT95 of 20 hours or more, and the device lifetime is approximately twice as long as that of the OLED device of Comparative Example 1B, which has an LT95 of 10 hours.

### <Evaluation 2 of OLED Device>

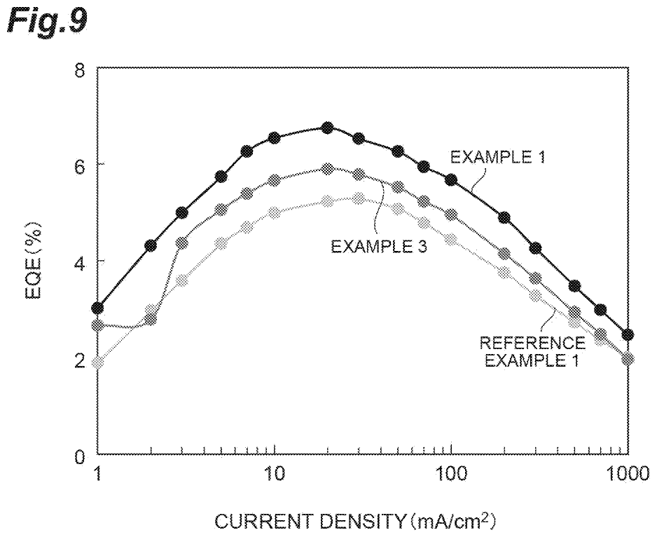

The external quantum efficiency (EQE) of the OLED devices of Examples 1 and 3 and Reference Example 1 was measured using a calibrated high-sensitivity/broadband spectrometer (AvaSpec-UV/VIS/NIR, manufactured by Avantes BV). The results are shown in FIG. 9.

As is clear from FIG. 9, the light-emission efficiency is the highest when the film thickness of the intermediate layer is 2 nm.

### Example 4

An MoO₃ hole transport layer (10 nm), a perylene layer (non-doped) (50 nm), an NDI-HF layer (50 nm), an LiF electron injection layer (3 nm), an MoO₃ electron injection layer (3 nm), and an Al electrode (100 nm) were thermally evaporated in this order in a high-vacuum state (about 10⁻⁵ Pa) in a vacuum evaporation system onto a glass substrate coated with indium tin oxide (ITO) (the thickness of ITO: 150 nm, a sheet resistance: 10.3 Ω/sq., manufactured by Techno Print Co., Ltd.). The resultant device was sealed with a glass substrate and an epoxy resin in a glove box to obtain an OLED device.

The obtained OLED device has the following configuration.
ITO electrode/MoO₃ hole transport layer/perylene layer (non-doped)/NDI-HF layer/LiF electron injection layer/MoO₃ electron injection layer/Al electrode

Note that, in the OLED device of Example 4, the “perylene layer (non-doped)” corresponds to the donor layer, and the NDI-HF layer corresponds to the acceptor layer.

### Comparative Example 2

An OLED device was prepared in a similar manner as in Example 4 except that a PTCDI-C₈ layer (50 nm) was formed by thermal evaporation instead of the NDI-HF layer. The OLED device has the following configuration.

ITO electrode/MoO₃ hole transport layer/perylene layer (non-doped)/PTCDI-C8 layer/LiF electron injection layer/MoO₃ electron injection layer/Al electrode

### <Evaluation 3 of OLED Device>

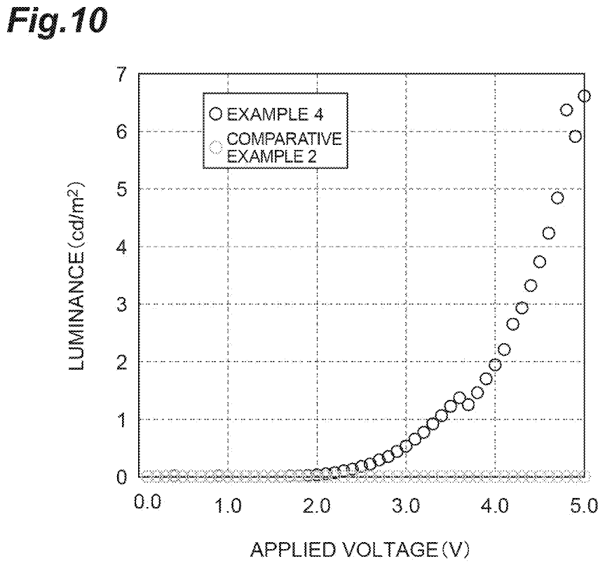

Applied voltage-luminance characteristics of the OLED devices of Example 4 and Comparative Example 2 were measured using a source measure unit (B2902A, manufactured by Keysight Technologies) and a luminance meter (BM-9, manufactured by TOPCOM CORPORATION). The results are shown in FIG. 10.

As is clear from FIG. 10, the OLED device of Example 4 is excellent in luminance in comparison to the OLED device of Comparative Example 2.

### REFERENCE SIGNS LIST

1: acceptor layer, 2: donor layer, 3: first electrode, 4: second electrode, 10, 100: OLED device, 11: glass substrate, 12: ITO electrode, 13: MoO₃ hole transport layer, 14: donor layer, 14A, 14C: rubrene (non-doped) layer, 14B: rubrene (doped with DBP) layer, 15: NDI-HF layer, 16: LiF electron injection layer, 17: MoO₃ electron injection layer, 18: Al electrode.

1. An OLED device comprising a pair of electrodes and a layered structure sandwiched between the electrodes,
wherein the layered structure has an acceptor layer and a donor layer forming an interface with the acceptor layer,
the acceptor layer contains an acceptor molecule,
the donor layer contains a donor molecule that causes triplet-triplet annihilation,
a HOMO level of the acceptor molecule is lower than a HOMO level of the donor molecule, and a LUMO level of the acceptor molecule is lower than a LUMO level of the donor molecule,
an excited triplet level of the donor molecule is smaller than a value obtained by adding 0.1 eV to an energy difference between the HOMO level of the donor molecule and the LUMO level of the acceptor molecule,
an excited triplet level of the acceptor molecule is larger than the energy difference between the HOMO level of the donor molecule and the LUMO level of the acceptor molecule, and
the energy difference between the HOMO level of the donor molecule and the LUMO level of the acceptor molecule is smaller than an energy difference between the HOMO level and the LUMO level of the donor molecule by 0.4 eV or more,
provided that a case is excluded where the acceptor layer is composed of an acceptor molecule represented by the following formula (A), the donor layer is composed of a first layer forming an interface with the acceptor layer and a second layer laminated on the first layer, the first layer is composed of a donor molecule represented by the following formula (B) and a dopant molecule represented by the following formula (C), and the second layer is composed of a donor molecule represented by the following formula (B):

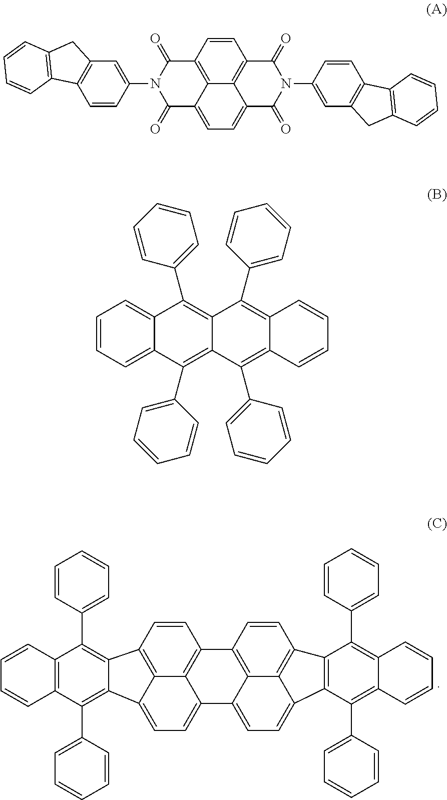

2. The OLED device according to claim 1, wherein the excited triplet level of the donor molecule is equal to or smaller than the energy difference between the HOMO level of the donor molecule and the LUMO level of the acceptor molecule.

3. The OLED device according to claim 1, wherein the donor layer further contains a dopant molecule.

4. The OLED device according to claim 2, wherein the donor layer further contains a dopant molecule.

5. The OLED device according to claim 1,
wherein the donor layer has a first layer forming an interface with the acceptor layer and a second layer laminated on the first layer,
the first layer contains a donor molecule that causes triplet-triplet annihilation, and
the second layer contains the donor molecule and a dopant molecule.

6. The OLED device according to claim 2,
wherein the donor layer has a first layer forming an interface with the acceptor layer and a second layer laminated on the first layer,
the first layer contains a donor molecule that causes triplet-triplet annihilation, and
the second layer contains the donor molecule and a dopant molecule.

7. The OLED device according to claim 1,
wherein the donor layer has a first layer forming an interface with the acceptor layer, a second layer laminated on the first layer, and a third layer laminated on the second layer,
the first layer contains a donor molecule that causes triplet-triplet annihilation,
the second layer contains the donor molecule and a dopant molecule, and
the third layer contains the donor molecule.

8. The OLED device according to claim 2,
wherein the donor layer has a first layer forming an interface with the acceptor layer, a second layer laminated on the first layer, and a third layer laminated on the second layer,
the first layer contains a donor molecule that causes triplet-triplet annihilation,
the second layer contains the donor molecule and a dopant molecule, and
the third layer contains the donor molecule.
